(19) United States
(12) Patent Application Publication
(10) Pub. No.: US 2006/0129213 A1
(43) Pub. Date: Jun. 15, 2006

(54) Method of sterilization of human and animal males

(76) Inventor: Fima Goldin, Brooklyn, NY (US)

Correspondence Address: Ilya Zborovsky, 6 Schoolhouse Way, Dix Hills, NY, 11746, US

(21) Appl. No.: 11/010,144

(22) Filed: Dec. 13, 2004

(51) Int. Cl. A61F 2/00

(52) U.S. Cl. 607/101

(57) ABSTRACT

A method of sterilization of human and animal males has the steps of acting on testicles with a thermal energy, and providing the thermal energy by a microwave radiation with a radiator which is located at a distance from the testicles and supplies the radiation to the testicles so as to heat the testicles to a temperature which provides their sterilization.

### BACKGROUND OF THE INVENTION

The present invention relates to a method of sterilization of human and animal males.

It is known to sterilize humans and animals to depress (temporarily or permanently). Such methods include hormonal and chemical contraceptives, surgical and other methods. The above listed methods can be further improved.

### SUMMARY OF THE INVENTION

Accordingly, it is an object of the present invention to provide a method of sterilization of human and animal males, which is a further improvement of the existing methods.

In keeping with these objects and with others which will become apparent hereinafter, one feature of the present invention resides, briefly stated, in a method of sterilization of human and animal males, comprising the steps of acting on testicles with a thermal energy; and providing the thermal energy by a microwave radiation with a radiation source which is located at a distance from the testicles and supplies the radiation to the testicles so as to heat the testicles to a temperature which provides their sterilization.

It is known that the temperature of testicles must be always several degrees (3°-4°) lower than the temperature of body of the human body. Therefore, when a temperature of the testicles is increased by 4-5° C., the reproductive function of the human or animal is depressed temporarily or permanently.

When the method is performed in accordance with the present invention, it is fast, convenient and efficient due to the fact that the testicles temperature is increased by a microwave radiation source which is remote from the testicles.

The novel features which are considered as characteristic for the present invention are set forth in particular in the appended claims. the invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a view schematically showing a device for carrying out a method for sterilization of human and animal males; and

FIG. 2 is a view showing a further modification of the device shown in FIG. 1.

### DESCRIPTION OF THE PREFERRED EMBODIMENTS

In accordance with the present invention, in order to provide sterilization of human or animal males, testicles of a human being or an animal, which are identified with reference numeral 1 are subjected to the action of a thermal energy so as to increase the temperature of the testacles by at least 4-5° C.

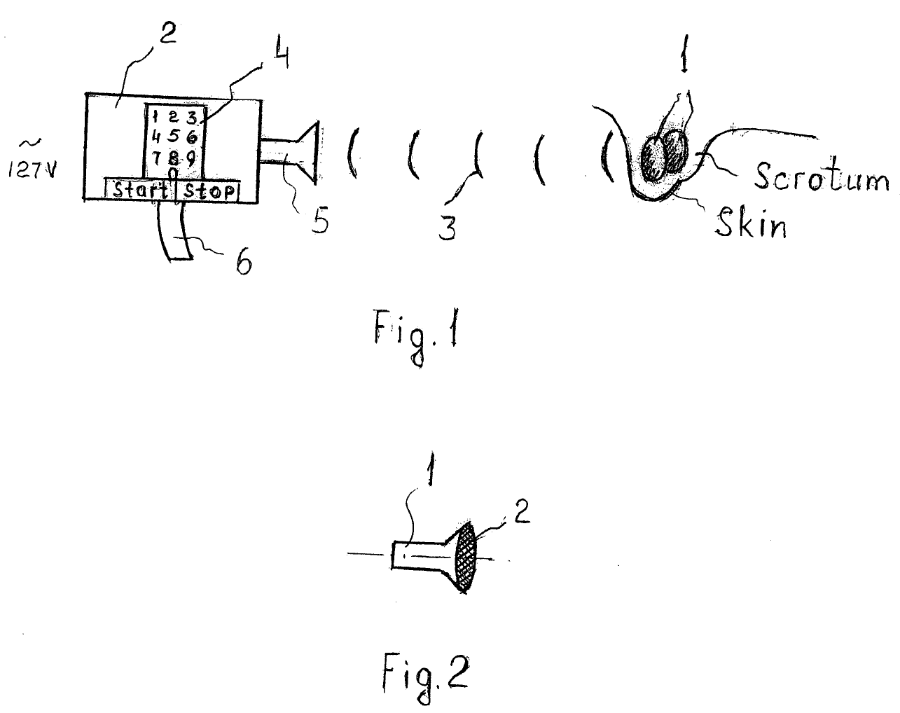

For this purpose, a microwave energy is utilized, which is generated for example by an SHF or microwave generator identified with reference numeral 2 in FIG. 1. The thusly formed generator emits waves of superhigh frequency as identified with reference numeral 3, and a beam of the radiation is directed toward the testicles 1. The radiation is selected so that the temperature of the testicles is increased by at least 4-5° C.

In accordance with a further feature of the present invention, a timer 4 is provided by the generator 2 so as to adjust a time of operation of the generator 2, which is necessary for heating the testacles 1 to a predetermined required temperature. Thus, the inventive process is time-adjustable.

An antenna 5 is further provided for focusing of SHF waves 3 in FIG. 1, which carry the microwave energy. As a result heat is directed only to the testacles 1 without affecting adjacent parts of the body of a human being or an animal.

The time which is adjusted by the timer 4 can be from several seconds to several minutes, since the time necessary for heating the testacles 1 for human beings and for different animals is different. When a button “start” of the generator 2 is turned on, the generated SHF waves 3 which carry the microwave energy are directed to the testicles 1 so as heat them and cause a temporary sterilization of a human being male or an animal male. The action on the testicles 1 can be selected so as to provide a permanent sterilization as well.

The method in accordance with the present invention is safe, it is inexpensive since it does not involve any medications, it can be used for human beings as well as for domesticated animals, such as for example cats, dogs, etc., for wild animals, for agricultural animals such as horses, bulls, pigs, sheep, etc.

In accordance with another feature of the present invention an antenna which is identified with FIG. 2 with reference numeral 11 is provided with an additional focusing element formed for example a lens, for accurate focusing of the microwave radiation to the testicles. Reference numeral 6 in FIG. 1 identifies a handle which can be grasped by a user for convenient manipulation with the generator 2.

It will be understood that each of the elements described above, or two or more together, may also find a useful application in other types of constructions differing from the types described above.

While the invention has been illustrated and described as embodied in, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of the invention.

What is claimed as new and desired to be protected by Letters Patent is set forth in the appended claims.

1. A method of sterilization of human and animal males, comprising the steps of acting on testicles with a thermal energy; and providing the thermal energy by a microwave radiation with a radiator which is located at a distance from the testicles and supplies the radiation to the testicles so as to heat the testicles to a temperature which provides their sterilization.

2. A method as defined in claim 1; and further comprising the steps of focusing microwave waves from the radiator toward the testacles.

3. A method as defined in claim 1; and further comprising the steps of adjusting a time of radiation of the testicles with the microwave waves.

4. A method as defined in claim 1; and further comprising the steps of providing an accurate focusing of the microwaves emitted by the microwave radiator with the use of a focusing lens.
